SEQUENCE LISTING (2 sequences)

SEQ ID NO: 1 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 2 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

(19) United States
(12) Patent Application Publication
(10) Pub. No.: US 2015/0250770 A1
(43) Pub. Date: Sep. 10, 2015

(54) LKB1 AS PREDICTIVE MARKER OF EVEROLIMUS EFFICACY IN BREAST CANCER

(71) Applicants: CENTRE LEON BERARD, Lyon (FR); INSTITUT NATIONAL DE LA SANTE ET DE LA RECHERCHE MEDICALE (INSERM), Paris (FR); UNIVERSITE CLAUDE BERNARD LYON 1, Villeurbanne (FR)

(72) Inventors: Isabelle Treilleux, Lyon (FR); Thomas Bachelot, Bron (FR); Muriel Le Romancer-Cherifi, Sainte Foy Les Lyon (FR)

(73) Assignees: CENTRE LEON BERARD, Lyon (FR); UNIVERSITE CLAUDE BERNARD LYON 1, Villeurbanne (FR); INSTITUT NATIONAL DE LA SANTE ET DE LA RECHERCHE MEDICALE (INSERM), Paris (FR)

(21) Appl. No.: 14/411,291

(22) PCT Filed: Jul. 1, 2013
(86) PCT No.: PCT/EP2013/063791

§ 371 Date: Dec. 24, 2014

Related U.S. Application Data
(60) Provisional application No. 61/666,451, filed on Jun. 29, 2012.

(30) Foreign Application Priority Data
Jun. 29, 2012 (EP) 12305769.7

(51) Int. Cl. A61K 31/436; A61K 31/138; G01N 33/574

(52) U.S. Cl. CPC A61K 31/436; G01N 33/57415; A61K 31/138; G01N 2333/912

(57) ABSTRACT

Method for identifying a breast cancer likely to respond to treatment with everolimus comprising the following steps: Measuring the level of expression of the LKB 1 gene in a breast tumour sample previously taken from a breast cancer patient, Classifying the breast cancer as likely to respond to treatment with everolimus if the LKB1 gene is under-expressed in said breast tumour sample.

The present invention relates to methods for predicting response to everolimus therapy in breast cancer patients.

The mTOR inhibitor everolimus has recently been shown to be able to significantly increase time to progression for metastatic breast cancer patient with ER+/HER2− disease that have progressed after aromatase inhibitor (AI) treatment. The TAMRAD trial was a “proof of concept” randomized phase 2 study that was first reported in 2010. 111 patients were included, time-to-progression (TTP) increased to 8.6 months with tamoxifen-everolimus from 4.5 months with tamoxifen (Bachelot T, 2012). These results were confirmed by the large BOLEROII registration trial in which 724 patients were randomized 2:1 to everolimus and exemestane versus exemestane and placebo. The primary end-point was progression free survival (PFS), based on local investigator assessment. It was increased from 2.8 months to 6.9 months with the addition of everolimus (hazard ratio=0.43 [95% Cl=0.35-0.54]; P<0.001) (Baselga 2012). In both trials, toxicities were significant, with fatigue (72% v 53% with tamoxifen), stomatitis (56% v 7%), rash (44% v 7%), anorexia (43% v 18%), and diarrhea (39% v 11%) being the most prevalent. In the TAM RAD study, 20% of the patients had to decrease the dose of everolimus, and 20% had to stop it due to toxicities (Bachelot T, 2012).

Unfortunately, at the present time, there is no biological marker that could select patients that are the more likely to benefit from the addition of everolimus to hormone therapy. Such a marker would allow increasing the therapeutic ratio of everolimus, which could be of high importance for its future development in the adjuvant setting.

The target of everolimus is the serine-threonine kinase mTOR (mammalian target of rapamycin). mTOR is a master regulator of protein synthesis, and plays important roles in other biological processes that support cell growth and survival, such as angiogenesis and autophagy. The prototypic mechanism for mTOR regulation by mitogenic signals is activation by the PI3K/Akt pathway. But other proteins can regulate mTOR, particularly the tumour suppressor LKB1 (STK11 NM_{—}000455) inhibits mTOR activity (for a review see Shackelford and Shaw 2009). In fact, LKB1 activates AMPK (AMP-activated serine/threonine protein kinase) through phosphorylation, and then AMPK directly phosphorylates 2 subunits of MTOR inhibiting its activity and phosphorylation of its downstream targets: 4EBP1 and S6kinase, proteins involved in protein synthesis.

The role of LKB1 as tumor suppressor as well as its low expression in tumors of different types of cancers, including breast cancer is known in the art (Fenton & al., 2006; Shen & al., 2002; Van Veelen & al., 2011). WO 2010/14486 discloses a method for evaluating the sensitivity of a patient having phakomatoses or hamartoma disease to a treatment with a mTOR inhibitor, by assessing the expression of several markers, including LKB1. Although it is mentioned that phakomatoses may be associated with breast cancer, no indication is made in this patent application that low expression of LKB1 may be used as a marker for selecting patients that are the more likely to benefit from the addition of everolimus to hormone therapy.

The present invention reports results of translational studies within the TAMRAD trial which show that low levels of the LKB1 protein, as assessed by imuno-histo-chemistry (IHC), can select patients whose tumour will be sensitive to everolimus.

### SUMMARY

The present invention relates to a composition comprising everolimus for use in treatment of a breast cancer patient, wherein said use comprises the following steps:
a) Selecting a breast cancer patient having a breast cancer tumour in which the LKB1 gene is under-expressed,
b) Administering to said patient a therapeutically effective amount of everolimus.

Preferably, in step a) said breast cancer tumour is a primary breast cancer tumour.

Preferably, the breast cancer patient has previously been classified as a ER+/HER2.

Preferably, the breast cancer patient has previously undergone breast surgery to remove a breast tumour.

Preferably, in step b) everolimus is administered in combination with hormone therapy.

Preferably, in step b) everolimus is administered in an adjuvant setting and/or in a metastatic setting.

Preferably, in step a) the level of expression of the LKB1 gene is lower than the median level of expression of the LKB1 gene in breast cancer tumours.

Another object of the present invention is a method for identifying a breast cancer likely to respond to treatment with everolimus comprising the following steps:
a) Measuring the level of expression of the LKB1 gene in a breast tumour sample previously taken from a breast cancer patient,
b) Classifying the breast cancer as likely to respond to treatment with everolimus if the LKB1 gene is under-expressed in said breast tumour sample.

Preferably, in step a) the breast tumour sample is a primary breast tumour sample.

Preferably, the breast cancer patient has previously been classified as ER+/HER2−.

Preferably, the breast cancer patient has previously undergone breast surgery to remove a breast tumour.

Preferably, in step b) the breast cancer is classified as likely to respond to treatment with everolimus in combination with hormone therapy.

Preferably, in step b) the breast cancer is classified as likely to respond to treatment with everolimus in an adjuvant setting and/or in a metastatic setting.

Preferably, in step b) the level of expression of the LKB1 gene is lower than the median level of expression of the LKB1 gene in breast cancer tumours.

### SEQUENCE LISTING

SEQ ID NO. 1: Human MTOR polypeptide (Human FKBP-rapamycin associated protein FRAP), Genbank L34075.1

SEQ ID NO. 2: Human LKB1 polypeptide (Human serine/threonine protein kinase), Genbank U63333.1

### DETAILED DESCRIPTION OF THE INVENTION

Treatment of breast cancer patients with everolimus is associated with toxicity and side effects for the patients. Moreover, not all breast cancer patients show an improvement in their OS and/or PFS after treatment with everolimus. There is a need for a biological maker identifying breast cancer patients more likely to benefit from a treatment with everolimus in particular in combination with hormone therapy.

The present invention provides a simple immunohistochemical assay identifying a subpopulation of breast cancer patients who are more likely to respond to treatment with everolimus.

In a first embodiment, the present invention relates to a composition comprising everolimus for use in treatment of a breast cancer patient, wherein said use comprises the following steps:
a) Selecting a breast cancer patient having a breast cancer tumour in which the LKB1 gene is under-expressed,
b) Administering to said patient a therapeutically effective amount of everolimus.

In a another embodiment, the present invention relates to a composition comprising everolimus for use in treatment of a breast cancer patient comprising administering to said breast cancer patient a therapeutically effective amount of everolimus wherein said patient is suffering from a breast cancer tumour in which the LKB1 gene is under-expressed.

Another object of the present invention is a method for treatment of a breast cancer patient comprising the following steps:
a) Obtaining a tumour sample from said patient,
b) Determining the level of expression of the LKB1 gene in said tumour sample,
a) Administering to said patient a therapeutically effective amount of everolimus if LKB1 is under-expressed in said tumour sample.

Another object of the present invention is a method for treatment of a breast cancer patient comprising the following steps:
a) Obtaining a tumour sample from said patient,
b) Determining the level of expression of the LKB1 gene in said tumour sample,
c) Administering to said patient a therapeutically effective amount of anti-cancer agent other than everolimus if LKB1 is under-expressed in said tumour sample.

In another embodiment, the present invention relates to a method for identifying a breast cancer likely to respond to treatment with everolimus comprising the following steps:
a) Measuring the level of expression of the LKB1 gene in a breast tumour sample previously taken from a breast cancer patient,
b) Classifying the breast cancer as likely to respond to treatment with everolimus if the LKB1 gene is under-expressed in said breast tumour sample.

In another embodiment, the present invention relates to a method for identifying a breast cancer patient having a tumour likely to respond to treatment with everolimus comprising the following steps:
a) Measuring the level of expression of the LKB1 gene in a tumour sample previously taken from said patient,
b) Classifying the breast cancer patient as having a tumour likely to respond to treatment with everolimus if the LKB1 gene is under-expressed in said tumour sample.

In another embodiment, the present invention relates to a method for selecting a treatment for a breast cancer patient having a tumour comprising the following steps:
a) Determining the level of expression of the LKB1 gene in a tumour sample previously taken from said patient,
b) Selecting everolimus as treatment for said breast cancer patient if the LKB1 gene is under-expressed in said tumour sample.

The term “cancer” refers to any disease in which a group of cells displays uncontrolled growth/proliferation, invasion and sometimes metastasis.

Preferably, the breast cancer patient classified or selected as having a tumour likely to respond to treatment with everolimus, is likely to have an improved PFS and/or an improved OS after treatment with everolimus.

Preferably, the methods of the present invention are in vitro methods.

The term “PFS” refers to Progression Free Survival and is defined as the percentage of patients staying free of disease progression during a period of time. In this case, the Kaplan-Meier curve represents the x % of patients staying free of disease progression after y amount of time.

The term “OS” refers to Overall Survival and is defined as the percentage of patients who survive after diagnosis of a cancer. In this case, the Kaplan Meier curve represents the x% of patients who survived after y amount of time.

The methods and compositions of the present invention are useful with respect to any cancer for which treatment with everolimus may be indicated. Cancers for which treatment with everolimus is approved or undergoing clinical trials include kidney cancer, pancreatic cancer, gastric cancer and lymphoma. Preferably, the methods and compositions of the present invention relate to breast cancer.

The term “everolimus” refers to the compound indifferently named RAD-001 or 42-O-(2-hydroxyethyl)rapamycin. Everolimus is an inhibitor of mTOR (mammalian target of rapamycin) encoded in humans by the MTOR gene (HGNC#3942; GenBank#L34075).

The present invention is based on an assay determining the expression level of the LKB1 gene.

The term “LKB1 gene” refers to the liver kinase B1 gene in humans also known as serine/threonine kinase 11 (STK11 gene, HGNC#11389, GenBank#U63333).

The term “sample” refers to any biological sample obtained/taken from a patient including a tissue sample, a cell sample or a tumour sample. In the present invention, the tumour sample contains cancer or tumour cells.

In preferred embodiments, the tumour sample of the present invention refers to a primary tumour sample.

The methods of the present invention rely on the determination of the level of expression of the human LKB1 gene in a sample taken from a cancer patient and in particular from a breast cancer patient. The level of expression of the LKB1 gene may be assessed by measuring the level of expression of the mRNA or of the polypeptide/protein encoded by the LKB1 gene.

In preferred embodiments, the level of expression of the LKB1 gene is determined by assessing the level of expression of the protein encoded by the LKB1 gene. In the present invention, the level of expression of the LKB1 gene may be measured quantitatively or semi-quantitatively. Advantageously, the level of expression of the LKB1 gene is determined in comparison with a control sample.

Any appropriate method known to the skilled person may be used to determine the level of expression of the LKB1 gene. At the mRNA level, expression of the LKB1 gene may for example be determined by RT-PCR, by Northern blotting or by hybridization to an immobilized probe. At the protein level, expression of the LKB1 gene may for example be determined using anti-LKB1 antibodies. Preferred methods include immunohistochemical methods such as immunostaining.

In a preferred embodiment, the level of expression of the LKB1 gene is determined on sections of tumour tissue by immunohistochemistry analysis with LKB1 antibodies. The level of expression of the LKB1 gene may preferably be determined semi-quantitatively using a complete histological score that considers both the staining intensity and the percentage of cells stained at a specific range of intensities. In these methods, sections of tumour samples are analysed by microscopy after contacting the sections with a labelled antibody binding specifically to LKB1.

In the methods of the present invention, the level of expression of the LKB1 gene is preferably compared to a control sample. In some embodiments, the control sample is the median level of expression of the LKB1 gene observed in a healthy breast tissue. More advantageously, the control sample is the median level of expression of LKB1 in tumour samples taken from patients having breast cancers and more preferably in primary breast tumor samples. In another embodiment, the control sample is the median level of expression of the LKB1 gene in ER+/HER2− breast tumours and more preferably in ER+/HER2− primary breast tumours.

In the present invention, the breast cancer patient has preferably been classified as ER+/HER2−.ER+ and HER2− are conventional markers of breast cancer based on the presence or absence of the estrogen receptor (ER) and of over expression or not of the human epidermal growth factor receptor 2 (HER2/ErbB-2). Generally, ER+/ HER2− breast cancers are classified as having a “better”/“good” prognosis. Receptor status may classically be determined by immunohistochemistry and/or detection of ERBB2 amplification according to well-known methods. Detection of ERBB2 amplification is usually carried out by ISH (in situ hybridization). Breast cancers of the ER+and/or HER2− subclass are considered as having a “favorable” prognosis although a number of these patients will experience a recurrence of their breast cancer.

Breast tumours or breast cancer patients belonging to the ER+/ HER2- subclass are usually selected or are considered suitable for hormone/endocrine therapy.

In the present invention the breast cancer patient may previously have undergone surgery for breast cancer to remove breast tumour.

In breast cancer, everolimus is typically used in combination with hormone/endocrine therapy. Hormone therapy may be used to help reduce the risk of the cancer coming back after surgery, but it may also be used for breast cancer that has spread or come back after treatment.

In hormone-receptor positive cancers, the hormone estrogen promotes the growth of breast cancer cells. Hormone therapy aims to block the effect of estrogen/progesterone or lower its levels in order to treat breast cancer. Some hormonal treatments are for example targeted at the estrogen receptor (ER). Hormone therapeutic drugs include compounds which block the estrogen receptor such as tamoxifen and fulvestrant. Hormone therapeutic drugs also include aromatase inhibitors blocking the synthesis of estrogen such as exemestane, anastrozole and letrozole.

The invention relates to a combination product for its use together or separately, simultaneously or in sequence, comprising everolimus and at least one hormone therapeutic drug to block the effect of estrogen/progesterone or lower its levels in the treatment of breast cancer in a patient, wherein the breast cancer patient is selected as having a breast cancer tumour in which the LKB1 gene is under-expressed. Said breast cancer tumour is particularly a primary breast cancer tumour and/or the breast cancer patient has been classified as a ER+/HER2−, and/or the breast cancer patient has previously undergone breast surgery to remove a breast tumour.

In the present invention everolimus may be administered in combination with therapeutic drugs blocking the estrogen receptor or in combination with aromatase inhibitors. In preferred embodiments, everolimus is administered in combination with tamoxifen or in combination with exemestane.

Everolimus may be administered in a metastatic setting or in an adjuvant setting. Adjuvant therapy is defined as a treatment given after the primary therapy to prevent that the cancer will come back or spread. Adjuvant therapy is typically applied after breast cancer surgery.

In the present invention, breast cancer patients are selected which are more likely to respond to treatment with everolimus. The identification of breast tumours/breast cancer patients which are more likely to benefit from treatment with Everolimus promotes a broader use of Everolimus in an adjuvant setting.

### EXAMPLES

### Methods

### The TAMRAD Study (Bachelot et al., 2012)

The TAMRAD study is a multicenter, open-label, phase II study. Key inclusion criteria were postmenopausal females, age ≧18 years, hormone receptor-positive and human epidermal growth factor receptor type 2 (HER2)-negative mBC not amenable to curative surgery or radiotherapy, and previous treatment with AIs in the adjuvant or metastatic setting. Patients had to be experiencing progressive disease as assessed by the local investigator. Other inclusion criteria included presence of ≦1 evaluable lesion (target or nontarget) and Eastern Cooperative Oncology Group (ECOG) performance status ≦2, Patients could have previously received any chemotherapy and/or radiotherapy before inclusion, either in the adjuvant or the metastatic setting.

Patients were randomly assigned in a 1:1 ratio to receive tamoxifen 20 mg/day or tamoxifen 20 mg/day plus everolimus 10 mg/day. Study treatment continued until disease progression, intolerable toxicity, or patient decision. No crossover was planned.

The primary efficacy endpoint was the 6-month clinical benefit rate (CBR), defined as the percentage of all patients with a complete or partial response or stable disease at 6 months. Secondary endpoints included time to progression (TTP), overall survival (OS), objective response rate, and toxicity determined by AEs and laboratory measures.

Between March 2008 and May 2009, 111 women with mBC previously treated with AIs were randomly allocated to receive tamoxifen plus everolimus (n=54) or tamoxifen alone (n=57). Between-group baseline demographic and clinical characteristics were generally well balanced. Previous AI treatment occurred in 41% of patients in the adjuvant setting and 67% in the first-line metastatic setting. Adjuvant chemotherapy was used in 51% of patients; 25% received chemotherapy in the metastatic setting. Median (range) duration of follow-up was similar for tamoxifen-everolimus and tamoxifen: 23.7 months (2.6-32.7) and 24.2 months (0.9-36.2), respectively.

The CBR at 6 months in the ITT population was 61% [95% Cl, 47-74] among patients treated with tamoxifen-everolimus versus 42% [95% Cl, 29-56] among those treated with tamoxifen alone (exploratory P=0.045). Results in the per-protocol population were similar (59% [95% Cl, 44-73 ] vs 41% [27-56]). The median TTP in the ITT population increased to 8.6 months (95% Cl, 5.9-13.9) with tamoxifen-everolimus from 4.5 months (95% Cl, 3.6-8.7) with tamoxifen alone (exploratory P=0.002). This difference in TTP corresponded to a 46% reduction in the risk of progression associated with tamoxifen-everolimus (HR, 0.54; 95% Cl, 0.36-0.81). At the last update of OS in September 2011, 16 patients in the tamoxifen-everolimus and 31 patients in the tamoxifen group had died. Median OS was not reached with tamoxifen-everolimus and was 32.9 months with tamoxifen, which translated to a 55% reduction in the risk of death associated with combination therapy (HR, 0.45; 95% Cl, 0.24-0.81; exploratory P=0.007).

### Immunohistochemistry Analysis of LKB1 in the Primary Tumor

51 tumor blocs from primary tumors of patients included in the TAMRAD study were retrieved. Paraffin embedded breast tumors were serially sectioned at a thickness of 4 μm. After deparaffinization and rehydration, tissue sections were boiled in low pH buffer (Dako) using a PT Link at 97° C. for 30 minutes. For blocking endogenous peroxidases, the slides were incubated in 5% hydrogen peroxide in sterile water. The slides were then incubated at room temperature for one hour with the anti-LKB1 polyclonal rabbit antibody (Ref: Ab58786, Abcam, Cambridge, UK) diluted at 1/50. After rinsing in Phosphate Buffer Saline, the slides were incubated with a biotinylated secondary antibody bound to a streptavidin peroxidase conjugate (Envision Flex kit Ref: K800021-2, Dako, Trappes, France). Bound antibody was revealed by adding the substrate 3,3-diamino benzidine. Sections were counterstained with hematoxylin.

All slides were read by a single senior pathologist who was blinded to the clinical data. For comparison of the staining among tissues, samples were scored semi-quantitatively using a complete histological score (H score) that considered both the staining intensity and the percentage of cells stained at a specific range of intensities. For each case, the complete H score was defined as the products of the percentage of cells stained at a given staining intensity (0-100) and the staining intensity score (0, none; 1, weak, 2, moderate, and 3, intense). LKB1 expression was categorized as below or at (H score ≦90) or above (H score >90) the median H score value of 90.

### Statistical Analysis

TTP was defined as the time from the date of randomization to the date of first documented progression or death due to underlying cancer, and estimated using the Kaplan-Meier method. Survival curves were compared using the log-rank test. Cox proportional hazards regression models were used to estimate hazard ratios (HRs). Statistical analysis was done using SAS 9.3 (SAS institute, Cary N.C.).

### Results

Patients with low LKB1 derived a strong benefit from everolimus in combination with tamoxifen, whereas no benefit was found in patients with high LKB1. For the patients with LKB1 H Score below or at 90, time to progression increased to 13.9 months (95% Cl, 2.0-23.6) with tamoxifen-everolimus from 4.5 months (95% Cl, 1.8-9.4) with tamoxifen alone (P=0.03). This difference in the time to progression corresponded to a 64% reduction in the risk of progression associated with tamoxifen-everolimus (HR, 0.36; 95% Cl, 0.13-0.95). By contrast, in patients with H Score above 90, time to progression was not found to be increased with the addition of everolimus (7.5 months (95% Cl, 1.7-23.8). vs 5.5 months (95% Cl, 1.9-9.7, p=0.35).

### Conclusion

Our results shows that everolimus in combination with hormone therapy is mostly active for patients with low levels of LKB1 as assessed by IHC in primary tumours.

### REFERENCES

Bachelot T, Bourgier C, Cropet C, Ray-Coquard I, Ferrero J M, Freyer G, Abadie-Lacourtoisie S, Eymard J C, Debled M, Spaëth D, Legouffe E, Allouache D, El Kouri C, Pujade-Lauraine E. Randomized Phase II Trial of Everolimus in Combination With Tamoxifen in Patients With Hormone Receptor-Positive, Human Epidermal Growth Factor Receptor 2-Negative Metastatic Breast Cancer With Prior Exposure to Aromatase Inhibitors: A GINECO Study. J Clin Oncol. 2012 May 7.
Baselga J, Campone M, Piccart M, Burris H A 3rd, Rugo H S, Sahmoud T, Noguchi S, Gnant M, Pritchard K I, Lebrun F, Beck J T, Ito Y, Yardley D, Deleu I, Perez A Bachelot T, Vittori L, Xu Z, Mukhopadhyay P, Lebwohl D, Hortobagyi G N. Everolimus in postmenopausal hormone-receptor-positive advanced breast cancer. N Engl J Med. 2012 Feb 9; 366(6):520-9.
Fenton H, Carlile B, Montgomery E A, Carraway H, Herman J, Sahin F, Su G H, Argani P. LKB1 protein expression in human breast cancer. Appl Immunohistochem Mol Morphol. 2006 June, Vol. 14, No 2.
   Shackelford D B, Shaw R J. The LKB1-AMPK pathway: metabolism and growth control in tumour suppression. Nat Rev Cancer. 2009 Aug; 9(8):563-75.
   Shaw R J, Shackelford D, Vasquez D. Gastrointestinal disease or disorder imaging and treatment. WO 2010/14486.
   Shen Z, Wen X-F, Shen Z-Z, Shao Z-M. The tumor suppressor gene LKB1 is associated with prognosis in human breast carcinoma. Clinical Cancer Research. 2002 July. Vol. 8.
Van Veelen W, Korsse S E, Van de Laar L, Peppelenbosch M P. The long and winding road to rational treatment of cancer associated with LKB1/AMPK/TSC/mTORC1 signaling. Oncogene. 2011. 30.

1-14. (canceled)

15. A method for treating a breast cancer patient comprising the following steps:
a. Selecting a breast cancer patient having a breast cancer tumour in which the LKB 1 gene is under-expressed,
b. Administering to said patient a therapeutically effective amount of everolimus.

16. The method according to claim 15, wherein in step a) said breast cancer tumour is a primary breast cancer tumour.

17. The method according to claim 15, wherein the breast cancer patient has been classified as a ER+/HER2−.

18. The method according to claim 15, wherein the breast cancer patient has previously undergone breast surgery to remove a breast tumour.

19. The method according to claim 15, wherein in step b) everolimus is administered in combination with hormone therapy.

20. The method according to claim 15, wherein in step b) everolimus is administered in an adjuvant setting and/or in a metastatic setting.

21. The method according to claim 15, wherein in step a) the level of expression of the LKB 1 gene is lower than the median level of expression of the LKB 1 gene in breast cancer tumours.

22. A method for identifying a breast cancer likely to respond to treatment with everolimus comprising the following steps:
c. Measuring the level of expression of the LKB1 gene in a breast tumour sample previously taken from a breast cancer patient,
d. Classifying the breast cancer as likely to respond to treatment with everolimus if the LKB 1 gene is under- expressed in said breast tumour sample.

23. The method according to claim 22, wherein in step a) the breast tumour sample is a primary breast tumour sample.

24. The method according to 22, wherein the breast cancer patient has been classified as ER+/HER2−.

25. The method according to claim 22, wherein the breast cancer patient has previously undergone breast surgery to remove a breast tumour.

26. The method according to claim 22, wherein in step b) the breast cancer is classified as likely to respond to treatment with everolimus in combination with hormone therapy.

27. The method according to claim 22, wherein in step b) the breast cancer is classified as likely to respond to treatment with everolimus in an adjuvant setting and/or in a metastatic setting.

28. The method according to claim 22, wherein in step b) the level of expression of the LKB 1 gene is lower than the median level of expression of the LKB 1 gene in breast cancer tumours.
